(19) United States
(12) Patent Application Publication
(10) Pub. No.: US 2013/0008449 A1
(43) Pub. Date: Jan. 10, 2013

(54) FLEXIBLE MASK ATTACHMENT FOR A PATIENT INTERFACE DEVICE

(75) Inventors: Lance Ranard Busch, Trafford, PA (US); Peter Chi Fai Ho, Pittsburgh, PA (US); Elizabeth Powell Margaria, Pittsburgh, PA (US)

(73) Assignee: KONINKLIJKE PHILIPS ELECTRONICS N.V., EINDHOVEN (NL)

(21) Appl. No.: 13/638,013

(22) PCT Filed: Mar. 4, 2011
(86) PCT No.: PCT/IB11/50932

§ 371 Date: Sep. 28, 2012

Related U.S. Application Data
(60) Provisional application No. 61/319,986, filed on Apr. 1, 2010.

(51) Int. Cl. A61M 16/06

(52) U.S. Cl. 128/206.21

(57) ABSTRACT

A patient interface device (10) that includes a mask (12) and a headgear component (18) for attaching the patient interface device to the head of a patient. The headgear component includes a strap (20) and an attachment element (22, 40, 60, 80) provided between the mask and the strap. The attachment element has a mask attachment portion (24, 42, 62, 82) coupled to the mask, a strap attachment portion (26, 44, 64, 84) coupled to the strap, and a flexible linkage portion (28, 46, 66, 86) provided between the mask attachment portion and the strap attachment portion. The flexible linkage portion is more flexible than both the mask attachment portion and the strap attachment portion to enable the mask attachment element to flex, bend, and/or twist along multiple axes to conform to particular facial contours of the patient.

This patent application claims the priority benefit under 35 U.S.C. §119(e) of U.S. Provisional Application No. 61/319,986 filed on Apr. 1, 2010, the contents of which are herein incorporated by reference.

The present invention relates to patient interface devices for transporting a gas to and/or from an airway of a user, and in particular, to a flexible attachment element for coupling a headgear to a mask of a patient interface device that provides for adjustment in multiple planes.

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device including a mask component on the face of a patient. The mask component may be, without limitation, a nasal mask that covers the patient's nose, a nasal pillow/cushion having nasal prongs that are received within the patient's nares, a nasal/oral mask that covers the nose and mouth, or a full face mask that covers the patient's face. The patient interface device interfaces the ventilator or pressure support device with the airway of the patient, so that a flow of breathing gas can be delivered from the pressure/flow generating device to the airway of the patient. Furthermore, it is known to maintain such devices on the face of a patient by a headgear having one or more straps adapted to fit over/around the patient's head.

A number of smaller, “low profile” masks have been developed and commercially released within the past few years. For many such “low profile” masks, the headgear force vectors necessary to achieve a robust and stable seal against the face of the patient cut a straight line near the corners of a patient's eyes, which can be uncomfortable and distracting. In order to avoid this, many manufacturers incorporate non-adjustable stiffeners into the headgear, allowing for a “dog leg” shape to achieve the necessary force vector while also avoiding the eyes. However, because the human face is so varied in size and shape, such stiffeners often do not provide an optimum fit.

Accordingly, it is an object of the present invention to provide a patient interface device that overcomes the shortcomings of conventional such devices. This object is achieved according to one embodiment of the present invention by providing a patient interface device that includes a mask and a headgear component for attaching the patient interface device to the head of a patient. The headgear component includes a strap and an attachment element provided between the mask and the strap. The attachment element has a mask attachment portion coupled to the mask, a strap attachment portion coupled to the strap, and a flexible linkage portion provided between the mask attachment portion and the strap attachment portion. The flexible linkage portion is more flexible than both the mask attachment portion and the strap attachment portion to enable the mask attachment element to flex, bend, and/or twist along multiple axes to conform to particular facial contours of the patient.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

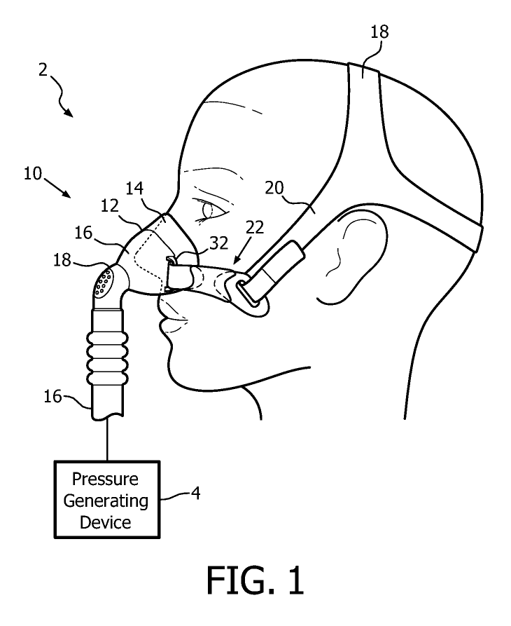

FIG. 1 is a schematic diagram of a system adapted to provide a regimen of respiratory therapy to a patient according to an exemplary embodiment of the invention;

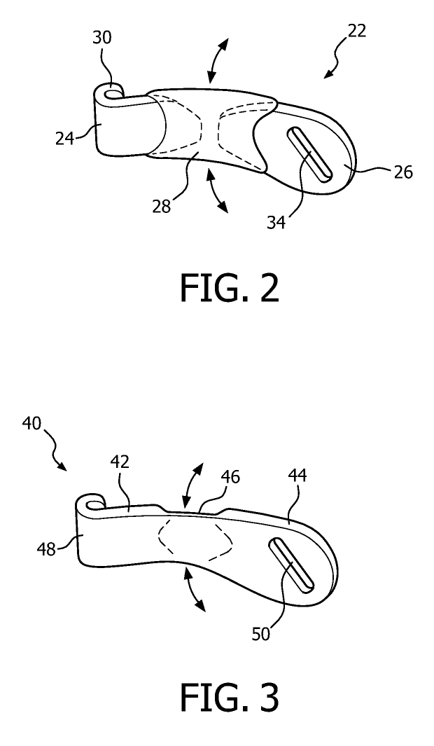

FIG. 2 is an isometric view of a mask attachment element according to one exemplary embodiment of the invention; and

FIGS. 3-5 are each isometric views of a mask attachment element according to alternative embodiments of the invention.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

System 2 adapted to provide a regimen of respiratory therapy to a patient according to an exemplary embodiment of the invention is generally shown in FIG. 1. System 2 includes a pressure generating device 4, a delivery conduit 6 coupled to elbow connector 8, and a patient interface device 10. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 10 through elbow connector 8. Delivery conduit 6, elbow connector 8 and patient interface device 10 are often collectively referred to as a patient circuit.

Patient interface device 10 includes a mask 12, which in the exemplary embodiment is nasal mask. However, any type of mask, such as a nasal/oral mask, a nasal pillow/cushion or a full face mask, which facilitates the delivery of the flow of breathing gas to the airway of a patient, may be used as mask 12 while remaining within the scope of the present invention. Mask 12 includes a cushion 14 coupled to a shell 16. Cushion 14 is made of a soft, flexible material, such as, without limitation, silicone, an appropriately soft thermoplastic elastomer, a closed cell foam, or any combination of such materials. In addition, shell 16 is made of a rigid or semi-rigid material such as, without limitation, polycarbonate or reinforced silicone. An opening in shell 16, to which elbow connector 8 is coupled, allows the flow of breathing gas from pressure generating device 4 to be communicated to an interior space defined by shell 16 and cushion 14, and then to the airway of a patient.

Patient interface device 10 also includes a headgear component 18, which in the illustrated embodiment is a two-point headgear. Headgear component 18 includes a first and a second strap 20, each of which is structured to be positioned on the side of the face of the patient above the patient's ear (only the left side strap 20 is shown). Headgear component 18 further includes a first and a second mask attachment element 22 (one on each side the head of the patient; only the left side mask attachment element 22 is shown). As described in greater detail herein, and as shown in FIG. 1, each mask attachment element 22 is provided to couple the end of one of the straps 20 to the respective side of mask 12. Of course, the present invention also contemplates providing only one mask attachment element 22 in the headgear component, for example, if so desired by the user.

FIG. 2 is an isometric view of mask attachment element 22 of the exemplary embodiment of FIG. 1. Mask attachment element 22 includes mask an attachment portion 24, a strap attachment portion 26, and a flexible linkage portion 28 provided between mask attachment portion 24 and strap attachment portion 26. Mask attachment portion 24 is made of a rigid or semi-rigid material, such as, without limitation, polycarbonate, reinforced silicone, high consistency silicone rubber (70 shore A and above), EVA copolymer, thermoplastic polyester elastomers (TPC-ET) (suitable examples of which are Hytrel® sold by DuPont, Riteflex® sold by Ticona), low-density polyethylene (LDPE), high-density polyethylene (HDPE), and polypropylene (PP).

Mask attachment portion 24 includes a hooked portion 30 at a first end thereof that is structured to be releasably engaged to a post 32 that is provided as part of each side of shell 16 of mask 12. Because hooked portion 30 is releasably engaged to a post 32, it provides for a quick release mechanism for headgear component 18. The present invention also contemplates providing other structures and/or elements for coupling mask attachment portion 24 to patient interface device 10. For example, a hook can be provided at strap attachment portion 26 or a component of ball-and-socket coupling can be provided at strap attachment portion. The present invention also contemplates that mask attachment portion 24 and patient interface device 10 can be formed as a unitary structure.

Strap attachment portion 26 is also made of a rigid or semi-rigid material, such as, without limitation, the rigid or semi-rigid materials described above, and includes slotted or looped portion 34 at a first end thereof that is structured to receive the end of a strap 20. The present invention contemplates providing other structures and/or elements for coupling a headgear strap 20 to strap attachment portion 26. For example, a hook can be provided at strap attachment portion 26 or a component of ball-and-socket coupling can be provided at strap attachment portion 26. The present invention also contemplates that strap attachment portion 26 and the associated headgear strap can be formed as a unitary structure.

Flexible linkage portion 28 is made of a material that is softer and more flexible than both mask attachment portion 24 and strap attachment portion 26. In the illustrated embodiment, flexible linkage portion 28 may be made a material such as, without limitation, a thermoplastic elastomer (TPE), silicone, rubber, silicone rubber, polyurethane (TPU), structural foam, or vinyl (PVC), and may be mechanically bonded to (e.g., by a strong friction fit or an adhesive) or overmolded onto the second ends of mask attachment portion 24 and strap attachment portion 26. When patient interface device 10 is donned by the patient, mask attachment element 22 is structured to fit under the cheekbone of the patient and flexible linkage portion 28 is able to flex, bend and/or twist along multiple axes to conform to the particular facial contours of the patient to provide an optimal fit. In addition, as seen in FIG. 1, mask attachment element 22 allows strap 20 to be moved away from the eyes of the patient.

FIG. 3 is an isometric view of mask attachment element 40 according to a first alternative embodiment. Mask attachment element 40 may be substituted for mask attachment element 22 in the embodiment of FIG. 1. Mask attachment element 40 includes a mask attachment portion 42, a strap attachment portion 44, and a flexible linkage portion 46 provided between mask attachment portion 42 and strap attachment portion 44. Mask attachment element 40 is a unitary structure made of a rigid or semi-rigid material, such as, without limitation, polycarbonate, high consistency silicone rubber (70 shore A and above), EVA copolymer, thermoplastic polyester elastomers (TPC-ET) (suitable examples of which are Hytrel® sold by DuPont, Riteflex® sold by Ticona), low-density polyethylene (LDPE), high-density polyethylene (HDPE), and polypropylene (PP).

As seen in FIG. 3, the thickness of flexible linkage portion 46 is smaller than the thickness of each of mask attachment portion 42 and strap attachment portion 44 such that flexible linkage portion 46 is able to readily flex, bend and/or twist while mask attachment portion 42 and strap attachment portion 44 remain substantially rigid or semi-rigid. The wall thickness section ratio of mask attachment portion 42 and strap attachment portion 44 to flexible linkage portion 46 will be highly dependent on the type of materials being used. For example, and without limitation, if a rigid material is used, a significant ratio such as 10 to 1 will be suitable, if semi rigid material is used, a suitable ratio will be somewhat lower, such as 5 to 1, and if a flexible material such as silicone is used, a suitable ratio may be 2 to 1.

In addition, mask attachment portion 42 includes a hooked portion 48 at a first end thereof that is structured to be releasably engaged to a post 32 that is provided as part of each side of shell 16 of mask 12. Strap attachment portion 44 includes a slotted/looped portion 50 at a first end thereof that is structured to receive the end of a strap 20. Thus, when patient interface device 10 having mask attachment element 40 is donned by the patient, mask attachment element 40 is structured to fit under the cheekbone of the patient and flexible linkage portion 46 is able to flex, bend and/or twist along multiple axes to conform to the particular facial contours of the patient to provide an optimal fit.

Both mask attachment element 22 and mask attachment element 40 are passive designs wherein each will return to substantially its original shape when forces applied thereto to cause it to flex, bend or twist are removed.

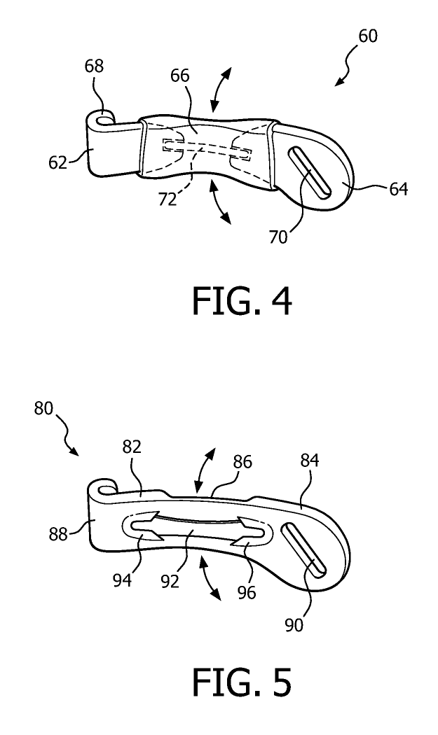

FIG. 4 is an isometric view of mask attachment element 60 according to a second alternative embodiment. Mask attachment element 60 may be substituted for mask attachment element 22 in the embodiment of FIG. 1. Mask attachment element 60 is an active adjustment design, meaning that it is conformable and will hold the shape into which is it flexed, bent or twisted. Mask attachment element 60 includes a mask attachment portion 62, a strap attachment portion 64, and a flexible linkage portion 66 provided between mask attachment portion 62 and strap attachment portion 64. Mask attachment portion 62 and strap attachment portion 64 are each made of a rigid or semi-rigid material as described elsewhere herein. Mask attachment portion 62 includes a hooked portion 68 at a first end thereof that is structured to be releasably engaged to a post 32 that is provided as part of each side of shell 16 of mask 12. Strap attachment portion 64 includes slotted/looped portion 70 at a first end thereof that is structured to receive the end of a strap 20.

Flexible linkage portion 66 is made of a material that is softer and more flexible than both mask attachment portion 62 and strap attachment portion 64. In the illustrated embodiment, flexible linkage portion 66 may be made a material such as, without limitation, a thermoplastic elastomer (TPE), silicone, rubber, silicone rubber, polyurethane (TPU), structural foam, or vinyl (PVC), and may be mechanically bonded to (e.g., by a strong friction fit or an adhesive) or overmolded onto the second ends of mask attachment portion 62 and strap attachment portion 64. In addition, as shown in FIG. 4, a pliable insert 72 is provided within flexible linkage portion 66 (e.g., by an overmolding process). Pliable insert 72 is structured to flex, bend and/or twist in response to applied forces and hold the resulting shape when the forces are removed.

Pliable insert 72 may be made of a common pliable metal or metallic compound, such as aluminum, copper, etc, a shape memory alloy (also referred to as SMA, smart metal, memory metal, memory alloy, muscle wire, smart alloy), which is an alloy that “remembers” its original, cold, forged shape, and which returns to that shape after being deformed by applying heat, a shape memory polymer, which has similar characteristics to a shape memory alloy, or an intermetallic compound, such as, without limitation, YAg, YCu, DyCu, CeAg, HoCu, NiTi, CuAlNi, or CuZnAlNi. When patient interface device 10 including mask attachment element 60 is donned by the patient, mask attachment element 60 is structured to fit under the cheekbone of the patient and flexible linkage portion 66 is able to flex, bend and/or twist along multiple axes to conform to the particular facial contours of the patient to provide an optimal fit. In addition, flexible linkage portion 66 will hold and maintain the resulting shape after the forces are removed.

FIG. 5 is an isometric view of a mask attachment element 80 according to a third alternative embodiment. Mask attachment element 80 may be substituted for mask attachment element 22 in the embodiment of FIG. 1. Mask attachment element 80 is also an active adjustment design, meaning that it is conformable and will hold the shape into which is it flexed, bent or twisted. Mask attachment element 80 includes a mask attachment portion 82, a strap attachment portion 84, and a flexible linkage portion 86 provided between mask attachment portion 82 and strap attachment portion 84. The main body of mask attachment element 80 is a unitary structure made of a rigid or semi-rigid material as described elsewhere herein.

As seen in FIG. 5, the thickness of flexible linkage portion 86 is smaller than the thickness of each of mask attachment portion 82 and strap attachment portion 84 such that flexible linkage portion 86 is able to readily flex, bend and/or twist while mask attachment portion 82 and strap attachment portion 84 remain substantially rigid or semi-rigid. The ratio of mask attachment portion 82 and strap attachment portion 84 to flexible linkage portion 86 may be as described elsewhere herein in connection with FIG. 3. Mask attachment portion 82 includes hooked portion 88 at a first end thereof that is structured to be releasably engaged to a post 32 that is provided as part of each side of shell 16 of mask 12, and strap attachment portion 84 includes slotted/looped portion 90 at a first end thereof that is structured to receive the end of a strap 20. In addition, mask attachment element 80 includes a pliable insert 92 that may be made of the same material as pliable insert 72 described elsewhere herein.

In the illustrated embodiment, pliable insert 92 is mechanically coupled to flexible linkage portion 86 by being received and held in pockets 94 and 96 provided on the surface thereof. Other mechanical coupling arrangements are also possible. Alternatively, pliable insert 92 may be provided within flexible linkage portion 86 by, for example, an overmolding process. When patient interface device 10 including mask attachment element 80 is donned by the patient, mask attachment element 80 is structured to fit under the cheekbone of the patient and flexible linkage portion 86 is able to flex, bend and/or twist along multiple axes to conform to the particular facial contours of the patient to provide an optimal fit. In addition, flexible linkage portion 86 will hold and maintain the resulting shape after the forces are removed.

It can thus be appreciated that the present invention provides a device that enables a headgear to provide a robust and stable seal against the face of the patient without interfering with the patient's eyes for a number of different facial sizes and shapes.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1-10. (canceled)

11. An attachment element for coupling a strap of a headgear component of a patient interface device (10) to a mask of the patient interface device, comprising:
a mask attachment portion structured to be coupled to the mask;
a strap attachment portion structured to be coupled to the strap;
a flexible linkage portion provided between the mask attachment portion and the strap attachment portion, wherein the flexible linkage portion is more flexible than both the mask attachment portion and the strap attachment portion; and
a pliable insert provided between the mask attachment portion and the strap attachment portion, wherein the pliable insert is structured to at least one of flex, bend or twist into a resulting shape in response to having forces applied thereto and to hold the resulting shape when the forces are removed.

12. The attachment element according to claim 11, wherein the mask attachment portion and the strap attachment portion are each made of a rigid or semi-rigid material.

13. The attachment element according to claim 12, wherein the rigid or semi-rigid material is selected from the group consisting of polycarbonate, reinforced silicone, high consistency silicone rubber (70 shore A and above), EVA copolymer, thermoplastic polyester elastomers (TPC-ET), low-density polyethylene (LDPE), high-density polyethylene (HDPE), and polypropylene (PP), and wherein the flexible linkage portion is made from a material selected from the group consisting of a thermoplastic elastomer (TPE), silicone, rubber, silicone rubber, polyurethane (TPU), structural foam, and vinyl (PVC).

14. The attachment element according to claim 11, wherein the mask attachment portion includes a hooked portion at a first end thereof that is structured to be releasably engaged to a post that is provided as part of the mask, and wherein the strap attachment portion includes a looped portion at a first end thereof that is structured to receive an end of the strap.

15. The attachment element according to claim 11, wherein the flexible linkage portion is structured to flex, bend, twist, or any combination thereof along multiple axes to conform to particular facial contours of the patient.

16. The attachment element according to claim 11, wherein the mask attachment portion, the strap attachment portion and the flexible linkage portion are separate components, and wherein a first end of the flexible linkage portion is attached to an end of the mask attachment portion and a second end of the flexible linkage portion is attached to an end of the strap attachment portion.

17. The attachment element according to claim 16, wherein the pliable insert is provided within the flexible linkage portion.

18. The attachment element according to claim 17, wherein the pliable insert is made of a material selected from the group consisting of a shape memory alloy, a shape memory polymer, a metal or metallic compound and an intermetallic compound.

19. The attachment element according to claim 17, wherein the mask attachment portion, the strap attachment portion and the flexible linkage portion are part of a unitary structure, wherein a thickness of the flexible linkage portion is less than a thickness of each of the mask attachment portion and the strap attachment portion, and wherein the thickness of the flexible linkage portion enables it to one or more of flex, bend and twist along multiple axes to conform to particular facial contours of the patient.

20. The attachment element according to claim 19, wherein the pliable insert is attached to a surface of the flexible linkage portion.

21. A patient interface device, comprising:
(a) a mask; and
(b) a headgear component for attaching the patient interface device to a head of a patient, the headgear component including a strap and an attachment element provided between the mask and the strap, wherein the attachment element comprises:
(1) a mask attachment portion structured to be coupled to the mask,
(2) a strap attachment portion structured to be coupled to the strap,
(3) a flexible linkage portion provided between the mask attachment portion and the strap attachment portion, wherein the flexible linkage portion is more flexible than both the mask attachment portion and the strap attachment portion, and
(4) a pliable insert provided between the mask attachment portion and the strap attachment portion, wherein the pliable insert is structured to at least one of flex, bend or twist into a resulting shape in response to having forces applied thereto and to hold the resulting shape when the forces are removed.

22. The patient interface device according to claim 21, wherein when the patient interface device is donned by the patient, the mask attachment element is structured to fit under a cheekbone of the patient and the strap is structured to extend along a side of the head of the patient between an eye and an ear of the patient.
